(19) United States
(12) Patent Application Publication
(10) Pub. No.: US 2010/0040703 A1
(43) Pub. Date: Feb. 18, 2010

(54) USE OF NITRIC OXIDE

(76) Inventors: Chris MILLER, North Vancouver (CA); Bruce MURRAY, Tofield (CA); Gilly REGEV-SHOSHANI, Vancouver (CA)

Correspondence Address: FASKEN MARTINEAU DUMOULIN LLP, 2900 - 550 Burrard Street, VANCOUVER, BC, V6C 0A3, CA

(21) Appl. No.: 12/540,335

(22) Filed: Aug. 12, 2009

Related U.S. Application Data
(60) Provisional application No. 61/088,656, filed on Aug. 13, 2008.

(51) Int. Cl. A61K 33/00; B01D 46/00; A61M 16/10; B65D 71/00; B05B 9/04

(52) U.S. Cl. 424/718; 96/226; 128/203.29; 206/572; 239/302

(57) ABSTRACT

The present invention relates to uses, compositions, devices, and methods involving the utilisation of nitric oxide to hinder, impede, inhibit, or prevent transmission of infectious viral particles to host cells.

### FIELD OF THE INVENTION

This invention relates to a method for hindering or preventing transmission of infectious viral particles.

### BACKGROUND OF THE INVENTION

Influenza is a highly infectious, acute respiratory illness caused by viruses that infect the respiratory tract. Influenza has been an intensive topic of scientific research and concern in the popular media of recent years. The highly pathogenic avian influenza viral strain, H5N1, infected 18 people in 1997, six of whom died from the infection. Similarly, an outbreak of the highly pathogenic H5N1 chicken virus in South-East Asia resulted in a high case-fatality rate in 2004. These and other incidents of influenza outbreaks have underscored the importance of developing methods of preventing the transmission of viral particles to new host cells.

Influenza virions are enveloped particles, of which there are three antigenic types: influenza A, B, and C. The influenza A viruses have been responsible for the major pandemics of influenza and are also the causative agents for most of the annual flu epidemics. Influenza A contains two major envelope proteins, haemagglutinin (HA) and neuraminidase (NA). The influenza A viruses are divided into subtypes based on the nature of these HA and NA glycoproteins. There are 15 HA and nine NA subtypes. Infection occurs by the binding of the HA glycoproteins to receptors on a host cell surface and subsequent fusion of the viral envelope with the host cell membrane, thereby permitting the RNA of the virus to enter the host cell, where it is replicated and ultimately results in the production of many new virus particles.

Influenza A passes from host to host in the form of inert particles and is commonly transmitted through aerosols spread into the environment by a sneezing or coughing infected individual. Such virus particles may be present and survive for extended periods of time on inanimate objects, and then may be transmitted to host cells. Virus particles in the air or on objects, attach to, and penetrate and infect cells in the aperture and open-wound areas of a subject's body as exemplified by the nose, mouth, eyes, abrasions, cuts, and sores.

Vaccination with either inactivated or attenuated virus preparations, remains the key method of influenza prevention by inducing a subject's immune system to develop virus-neutralizing antibodies in their system. Antiviral drugs have also been used in the treatment or prevention of influenza infection. Antiviral drugs approved for treatment or prophylaxis of influenza include amantadine, rimantadine, oseltamivir and zanamivir. These drugs interfere with specific steps in the replication cycle of the influenza virus, either at the level of virus entry or at the level of virus assembly release from the infected cell. However, viral resistance to amantadine and rimantadine has become problematic, and it is possible that resistance to oseltamivir could become a problem as well. Neither vaccination nor antiviral drug treatment is aimed at targeting a virus particle prior to its entry into a new host cell or subject. It is desirable to hinder or prevent air-borne transmission of infectious virus particles, i.e., virions, and transmission of surface-borne infectious virions to a host's susceptible cells and tissues. However, the currently available anti-viral drugs are not useful for hindering or prevention of infections of hosts by air-borne and/or surface-borne virions.

The currently available antiviral drugs interfere with certain steps in the replication cycle of the influenza virus, either at the level of virus entry or at the level of virus assembly release from the infected cell. The process of infecting susceptible host cells by infectious virions generally starts with the virions contacting the surface of the host cell, followed by attachment of one or more virions to the host cell's surface. The host cell subsequently engulfs the virion after which a channel is formed through the host cell's membrane. The virion's genetic material then spills into the endoplasm of the host's cell after which the viral replication processes are initiated.

Nitric oxide (NO) is produced in the endothelium tissue of the human body as part of normal physiological processes. NO is an endogenous vasodilator i.e., an agent that widens the internal diameter of blood vessels. NO is also useful for ameliorating the effects of physiological bronchial disorders exemplified by asthma, adult respiratory distress syndrome (ARDS) and chronic obstructive pulmonary disease (COPD). It is known that low concentrations of NO can be useful antibiotic treatments for various types of bacterial infections. NO has been shown to be an effective anti-microbial and/or microcidal agent for a broad range of microorganisms when applied in NO gas-releasing compositions and devices by causing a reduction in their intracellular detoxifying thiol levels. Furthermore, it appears that inhalation of low concentrations of NO into patients' airways can be useful antimicrobial treatments for various pulmonary diseases such as cystic fibrosis. It has also been suggested that NO has an inhibitory effect on the life cycle of the influenza virus. See, for example, Rimmelzwaan et. al., Journal of Virology; Vol. 73, No. 10; p. 8880-8883 (October 1999) and Akerstrom et. al, Journal of Virology; Vol. 79, No. 3; p. 1966-1969 (February 2005).

### SUMMARY OF THE INVENTION

Surprisingly it has been found that exposure to NO in their immediate environments significantly impairs the subsequent ability of infectious virions to attach to, penetrate into, and infect susceptible host cells. Furthermore, we have discovered that exposure of an infectious virion to NO following its release from an infected host cell affects the outer surface properties of the virion which hinders the virion's penetration and replication in a new host cell. It is believed that NO has broad spectrum effect may be used against any type of virus. In an embodiment of the present invention the viral target family is selected from those having envelopes. Examples of suitable target family's include herpesviridae, togaviridae, flavivirida, coronavirus, orthomyxoviridae, paramyxoviridae, filoviridae, retroviridae, hepadnaviruses, and the like. Examples of suitable target viruses include herpes simplex virus, rubella virus, hepatitis C, Yellow fever, SARS, influenza virus A/B/C, mumps, respiratory syncytial, Ebola virus, HIV, hepatitis B, and the like.

The present invention relates to uses, compositions, devices, and methods involving the utilisation of nitric oxide to hinder, impede, inhibit, or prevent transmission of infectious viral particles to host cells.

As used herein, the term “hindering, impeding, inhibiting, or preventing transmission of infectious viral particles” means that the probability of a certain virion successfully infecting a new host cell is reduced compared to regular pathogenic conditions.

### BRIEF DESCRIPTION OF THE DRAWINGS

The present invention will be described in conjunction with reference to the following drawings, in which:

FIG. 1 is a side view of an individual wearing an exemplary mask according to an exemplary embodiment of the present invention;

FIG. 2 is a close-up sectional side view of the exemplary mask shown in FIG. 1;

FIG. 3 is a close-up sectional side view of the exemplary mask shown in FIG. 1, fitted with an optional nasal canula inserted into a subject's nostril;

FIG. 4 is an exemplary illustration of an intermittent positive pressure breathing device cooperatable with the exemplary mask shown in FIG. 1, for infusing incoming or outgoing air with NO;

FIG. 5 is an exemplary illustration of the delivery of gaseous NO into an air-filter.

### DETAILED DESCRIPTION OF THE INVENTION

The scope of this invention is not limited to a presumption of a specific mode of action by the NO on infectious virions and it is possible that there is more than one mode by which NO affects virions. However, it is believed that one or more of the surface glycoprotein hemoagglutinin HA, M2 protein, ribonucleoprotein, neuraminidase among others on the outer surfaces of the virions interact with and/or are modified by NO molecules immediately upon contact. Exposure of a virion to NO results in the alteration of the virion, impairing it from binding to targets on the host cell surface and entering the new host cell. It is believed that, while viruses do not by themselves have thiol-based detoxification pathways, they may still be inherently more susceptible to reactive nitrogen species and nitrosactive stress. NO may inhibit a necessary constituent enzyme required for viral DNA synthesis, and therefore, inhibit viral replication. NO may also inhibit the replication of viruses early during the replication cycle, involving the synthesis of vRNA and mRNA encoding viral proteins. Further, it is believed that the NO molecule attacks the cysteine sites or nitrosylates the sulphur bonds in the HA glycoprotein on the surface of the virus. For example, the NO molecule may bind to the cysteine groups, thus altering the structure of HA and/or NA glycoproteins. NO may also bind to the outer surfaces of virions thereby preventing endocytotic engulfinent of virions by host cells. Alternatively, NO molecules may cause S-nitrosylation in protein molecules thereby altering the structure of HA and/or NA glycoproteins.

Regardless of the mechanism, it is believed that the virus is rapidly affected upon contact with NO molecules and exposure of a virion to NO can render the virus non-infectious and unable to enter a new host cell. It appears that the initial exposure of infectious virions to NO has the greatest effect on inactivation rather than the duration of virus exposure to NO. Effectiveness is thought to be related to the number of viral targets for NO on the surface of the virions while the time, or dose, of exposure of lesser importance.

The present invention relates to the use of nitric oxide for reducing the transmissibility of a virus. The NO may be in any suitable form. For example, the NO may be in gaseous form or it may be in solution.

The present invention may be used against any virus that has its transmissibility reduced by exposure to NO. In an embodiment, the present invention is targeted at orthomyxoviridae. The present invention has been found efficacious against the influenza virus. For example, the present invention may be used to limit the transmission of H1N1, H5N1, H3N2, or the like.

Exposure to high concentrations of NO may be toxic, especially exposure to NO in concentrations over 1000 ppm. Even lower levels of NO can be harmful if the time of exposure is relatively high. Accordingly, in one embodiment, the uses, compositions, devices, and methods of the present invention utilise the minimally effective dose of NO in order to achieve the desired reduction in viral transmissibility.

The present invention may utilise NO in any suitable form. In one embodiment NO is delivered in the form of a gas which, upon contact with a virion, inhibits viral transmissibility. The concentration of NO gas may be from about 1 ppm to about 1000 ppm, or from about 10 ppm to about 500 ppm, or from about 25 ppm to about 100 ppm.

The NO gas may be delivered in any suitable form. For example, the gas may be released from a non-pressurised or a pressurised canister into the desired location. Alternatively, the NO may be produced in-situ and released directly into the target location. The NO gas may be infused into the target location. For example, NO gas can be infused into devices, fabrics, materials, plastics, etc.

The NO may be delivered in solution with a suitable solvent. For example, saline treated with NO has been found to inhibit viral transmissibility. Therefore, it is within the scope of this invention to utilise NO in a solvent for inhibiting viral transmissibility. Any suitable solvent or mixture of solvents may be used herein. For example, NO is soluble in water and various alcohols such as methanol, ethanol, isopropanol, and the like. In an embodiment the solvent is water, for example, in the form of saline. One aspect of the present invention relates to an anti-viral composition comprising a nitric oxide solution as described herein.

In an embodiment the pH of the NO solution is from about 3 to about 6, or from about 3.5 to about 5.5.

In an embodiment the concentration of nitrites/nitrates in solution is from about 10 μM or greater, or from about 50 μM or greater, or from about 100 μM or greater, or from about 120 μM or greater. In an embodiment the concentration of nitrites/nitrates in solution is from 200 μM or less, or from about 190 μM or less, or from about 180 μM or less.

One aspect of the present invention involves the use of nitric oxide for inhibiting the transmissibility of a virus wherein nitric oxide is brought into contact with a locale that has been, or is at risk of being, exposed to a virus. As described above, the NO may be a gas or in solution.

In an embodiment of the present invention, an NO solution may be applied to surfaces, fabrics, or the like as a method of inhibiting the transmission of a virus. The NO solution may conveniently be in any suitable form but conveniently may be in the form of a spray or aerosol. The solution may be applied to patients infected with a virus in order to limit the transmissibility of the virus. Alternatively, the solution may be applied to subjects at risk of being infected by a virus.

One aspect of the present invention comprises a spray and/or aerosol device comprising a composition of nitric oxide. The device comprises an orifice and a reservoir wherein the reservoir communicates with the orifice and comprises a supply of nitric oxide. The NO may be in gaseous form or in solution. If a gas, the NO may be compressed. In an embodiment the NO is in solution such as a saline solution.

The present invention further comprises methods of treating an animals infected with a virus comprising applying nitric oxide to the animal. In certain embodiments the animal is a human or non-human mammal. In an embodiment the animal is a human. The nitric oxide may be applied as a gas or in solution.

The present invention further comprises methods of prophylactically treating an animals at risk of being infected with a virus said methods comprising applying nitric oxide to the animal. In certain embodiments the animal is a human or non-human mammal. In an embodiment the animal is a human. The nitric oxide may be applied as a gas or in solution.

An embodiment of the present invention comprises utilising a nasal spray of NO to limit the transmission of a virus via the nasal cavity of an animal such as a human. In an embodiment the present invention is utilised in the treatment or prevention of sinusitis, non-healing wounds, viral infections, or the like.

The present invention may be used for the prevention or treatment of virus infection and/or spread in an avian species. For example, the present invention may be used on commercial operations such as poultry farms for addressing viral infection or for preventing the same.

In an embodiment the present invention has an effect on the transmissibility of viruses after a single dose of NO. Alternatively multiple doses may be used.

In one aspect of the present invention, a controlled method is used to deliver an effective amount of NO to the airways of infected patients in which virions are present or prior to release into the environment prior to their entry into a new host cell. The present invention is thus advantageous to current methods of prophylaxis or treatment since it targets virus particles extracellularly while outside a host cell to inhibit infectivity of the virions. The NO may be in gaseous form or in solution.

The present invention relates to an anti-viral air-filter comprising a filter element and nitric oxide. The filter element may be in any suitable form but in an embodiment it is fibrous. In an embodiment the element is selected from fibrous woven or non-woven fabrics.

The air-filter may comprise a dischargeable supply of gaseous nitric oxide. In an embodiment the air-filter comprises a dischargeable supply of gaseous nitric oxide which periodically delivers nitric oxide in a concentration of at least about 25 parts per million.

The air-filter may comprise nitric oxide in the form of a solution as described herein.

In an embodiment the nitric oxide is applied to the filter element in the form of a solution, the solution having a pH of from about 3 to about 6 and a nitrite/nitrate concentration of from about 120 μM to about 190 μM.

The filter herein may be a facemask configured and sized to substantially cover the nose and mouth of the user and to be secured thereto.

The filter herein may be an air-duct filter configured and sized to fit in an air duct system.

The present invention further relates to an apparatus comprising a facemask configured to communicably cooperate with a supply of nitric oxide; a dischargeable supply of nitric oxide; and a device configured to controllably provide from the dischargeable supply of nitric oxide. In an embodiment the device delivers from about 100 ppm to about 200 ppm of nitric oxide. The supply of nitric oxide may optionally be demountably engagable with the facemask.

The facemasks of the present invention may be included in a kit comprising; a facemask configured and sized to substantially cover the nose and mouth of the user and to be secured thereto; a dischargeable supply of nitric oxide; the facemask and nitric oxide being sealably contained within a gas-impermeable container.

The facemasks herein may be configured and sized to substantially cover the nose and mouth of the user and to be secured thereto; the nitric oxide may be applied in the form of a solution.

An exemplary embodiment of the present invention relates to methods, systems and apparatuses for significantly impairing and/or preventing the ability of air-borne virions to subsequently infect a host wherein a subject is provided with a facial mask communicably cooperatable with a dischargeable source of NO gas or solution released into the atmosphere in the vicinity of the subject's mouth and nostrils. In an embodiment the discharge of the source of NO is controllable. In an embodiment the NO is delivered in the form of an aerolized solution. If infectious virions are present in the atmosphere, the subject's breathing may draw the virions through the atmosphere immediately adjacent the subject's respiratory orifices wherein the virions encounter NO molecules thus resulting in functional impairment of the virions' ability to infect new host cells. Such masks are also suitable for virus-infected hosts to wear when they are interacting with non-infected individuals. In this case, the infected hosts will be expelling infectious virions while they are breathing and during coughing and sneezing episodes. The infectious virions will pass through the NO-infused facial masks disclosed herein, and will be debilitated by the NO molecules, thereby reducing the infection risk to the uninfected hosts in the vicinity of the infected host. Virions already within the host's airways exposed to the NO molecules during inhaling will also have their infectivity potential reduced so that if they are expelled during exhalation, speaking or coughing, other host cells are less likely to become infected.

As used herein, the term “dischargeable” means to the that the NO source is able to release NO. The mechanism of release may be any suitable means including passive release such as diffusion or active release.

Any suitable mask may be used herein. For example, disposable masks comprising fibrous substrates selected from naturally derived materials such as cellulose fibres and/or synthetic polymeric fibres. Infusing such masks with NO gas or solution results in retention of NO molecules in the fibrous substrates. Other devices for delivery of NO molecules during mechanical ventilation have been described which may be used for this application when hosts are unable to breathe for themselves. For examples of suitable devices see U.S. Pat. No. 7,516,742; WO2009/036571; U.S. Prov. Pat. App. 61/043,639; which are herein incorporated by reference.

The present invention encompasses methods for infusing suitable facial masks with NO and kits comprising one or more NO-infused facial masks. In an embodiment the NO-infused masks are sealed in suitable gas-impermeable containers. It is within the scope of the present invention to provide kits comprising a plurality of disposable facial masks, a plurality of single-use quantities of NO gas or solution in suitable dispensers, and a device provided for temporarily sealably containing at least one facial mask with a NO-gas dispenser, wherein the device is configured for release of the NO into the temporarily sealed container to infuse the mask for a selected period of time. The device may be re-useable or alternatively, disposable. It may be suitable to configure the facial masks for demountable cooperative communication with pressurized gas canisters containing therein NO gas or solution. The pressurized gas canisters may be configured to controlling devices for controllable and manipulable delivery of NO into the facemasks at concentrations of about 600 ppm to about 1,500 ppm, wherein the NO concentration is immediately diluted to about 160 ppm by intermixing with the atmosphere about and within the facemask.

The present invention also relates to method of inhibiting the transmission of viral microorganisms, the method comprising the steps of providing a supply of nitric oxide; and contacting the viral microorganisms with the nitric oxide. For example, the method may comprise treating an animal having pathogenic viral microorganisms in its respiratory tract the treatment comprising delivering an amount of nitric oxide to the respiratory tract of said animal, the amount of nitric oxide being effective to inhibit the transmissibility of said pathogenic microorganisms.

The present methods may be used to inhibit the transmissibility of pathogenic influenza viruses.

In one embodiment the method of inhibiting the transmission of pathogenic influenza viruses comprises the steps of: providing a supply of nitric oxide gas; contacting the viral microorganisms with the nitric oxide; wherein the concentration of nitric oxide is from about 25 parts per million to about 100 parts per million.

In an embodiment the method of inhibiting the transmission of pathogenic influenza viruses comprises the steps of providing a supply of nitric oxide solution; contacting the viral microorganisms with the nitric oxide; wherein the concentration of nitric oxide is from about 120 μM to about 190 μM.

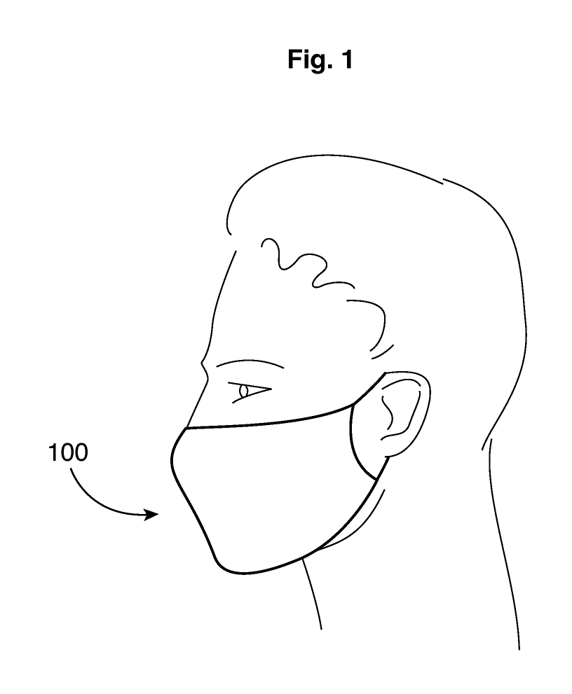

Referring now to the figures, FIG. 1 shows a side view of an individual wearing an exemplary face mask (100) according to the present invention.

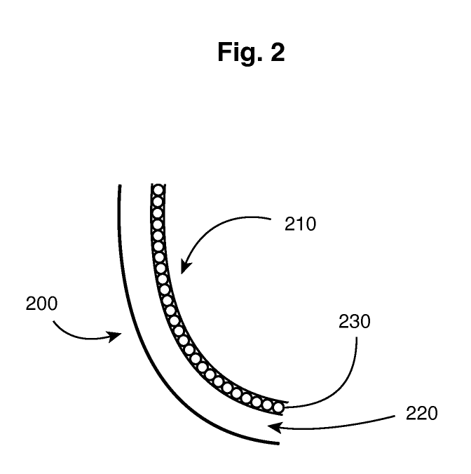

FIG. 2 is a close-up sectional side view of the exemplary mask shown in FIG. 1. The mask (200) has an interior face (210), a cavity (220) into which NO gas may be delivered, and a filter (230).

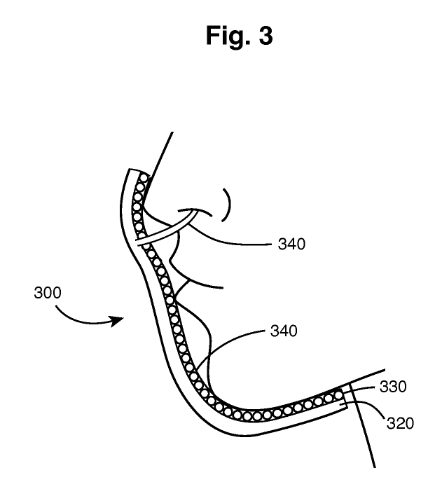

FIG. 3 is a close-up sectional side view of the exemplary mask shown in FIG. 1. The mask (300) has an interior face (310), a cavity (320) into which NO gas may be delivered, a filter (330), and is fitted with a nasal cannula (340) inserted into a subject's nostril.

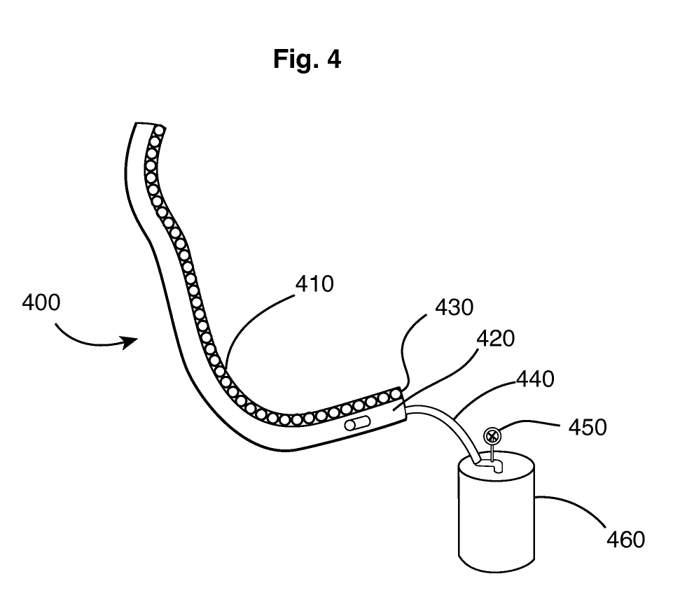

FIG. 4 shows a face mask (400) having an interior face (410), a cavity (420), a filter (430), a supply of NO gas (460) which is delivered to the cavity (420) via pipe (440), the flow being controlled by valve (450).

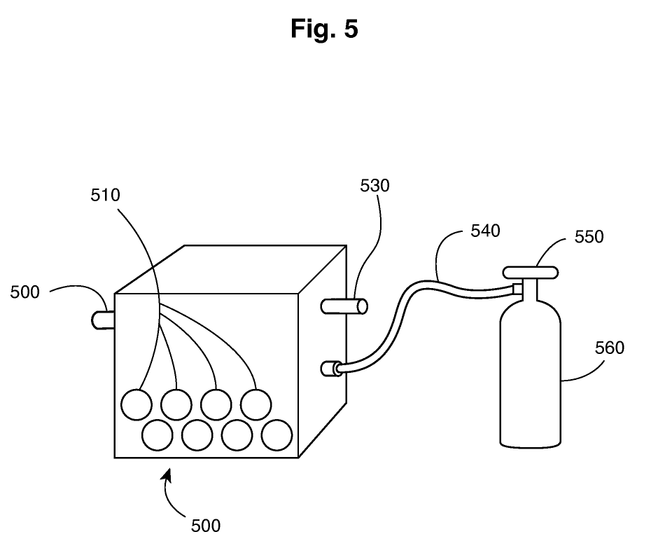

FIG. 5 is an exemplary illustration of an air-filter (500) which has a filter (410), an inflow pipe (520), an outflow pipe (530), a supply of NO gas (560) which is delivered via pipe (540), the flow being controlled by valve (550).

The present invention is described with reference to specific details, preferences, and examples of particular embodiments thereof. It is not intended that such details and examples be regarded as limitations upon the scope of the invention except insofar as and to the extent that they are included in the accompanying claims. As used in this specification and the appended claims, the singular forms “a,” “an,” and “the” include plural referents unless the content clearly dictates otherwise. Unless otherwise specified all documents referred to herein are incorporated by reference.

### Example 1

1 mL of 6×10⁵ plaque forming units (pfu) of a surrogate strain of influenza H3N2 were placed in 3 wells of a 6 well plate and exposed for 1, 2 and 3 hours to either 160 ppm gNO (Tx) or room air (control). At each exposure time a volume from each tray (1 mL from 3 wells=3 mL) was extracted and frozen at −70° C.

Madin-Darby Canine Kidney (MDCK) cells were grown in 6 well plates to a confluent monolayer. When cells were ready a 0.5 mL sample of each time point for treatment and control were inoculated into the cells and incubated on a shaker tray for 1 h at 37° C. The trays were then fixed with agar/media/trypsin and incubated at 37° C. for 3 days until plaques formed. The trays were then fixed with 4% formaldehyde and stained with crystal violet, then dried.

As Table 1 shows, influenza A virions that were exposed to 160 ppm NO before incubation with host cells were at least 80% less transmissible into host cells than the control group.

TABLE 1
Effect of treatment with NO-gas on Influenza A/Victoria/H3N2

| | Control | NO-treated |
|---|---|---|
| 0 mins | 250 ± 14 pfu/ml | 250 ± 14 pfu/ml |
| 60 mins | 255 ± 38 pfu/ml | 50 ± 11 pfu/ml |
| 120 mins | 266 ± 37 pfu/ml | 43 ± 10 pfu/ml |
| 180 mins | 270 ± 23 pfu/ml | 29 ± 14 pfu/ml |

### Example 2

50 ml of 10,000 ppm NO gas was injected into a sterile IV bag containing 50 ml of 0.9% saline solution. From stock of influenza A%Victoria/H3N2 an inoculum of 10⁷ virions was prepared in phosphate buffered saline (PBS).

0.5 ml of the inoculum was inoculated into the NO-containing saline. A further 0.5 ml of inoculum was inoculated into a 50 ml bag of 0.9% saline that had not been treated with NO. Additionally, 0.5 ml of inoculum was inoculated into a 50 ml bag of 0.9% saline that had been injected with 50 ml of air. Samples were drawn at 1, 3, 5, 10, 15, 45, 60, 120 and 180 minutes. The samples were then plated on 6 well trays with confluent MDCK cells.

A standard plaque assay was performed. After 2 days the plates were fixed and stained. The plaques were counted. The results are shown in Table 2 demonstrating that both control arms remained viable with no reduction in the number of plaques. No infectious units remained in any of the treatment arm samples.

TABLE 2
Effect of NO-treated saline on Influenza A/Victoria/H3N2

| | | | |
|---|---|---|---|
| 0 mins | 1.1 × 10⁵ ± 8.1 × | 1.1 × 10⁵ ± 8.1 × | 1.1 × 10⁵ ± 8.1 × |
| | 10⁴ pfu/ml | 10⁴ pfu/ml | 10⁴ pfu/ml |
| 1 min | 1.1 × 10⁵ ± 8.1 × | 1.1 × 10⁵ ± 8.1 × | 0 pfu/ml |
| | 10⁴ pfu/ml | 10⁴ pfu/ml | |
| 3 mins | 1.1 × 10⁵ ± 7.1 × | 1.1 × 10⁵ ± 7.9 × | 0 pfu/ml |
| | 10⁴ pfu/ml | 10⁴ pfu/ml | |
| 5 mins | 1.0 × 10⁵ ± 1.2 × | 1.0 × 10⁵ ± 4.3 × | 0 pfu/ml |
| | 10⁴ pfu/ml | 10⁴ pfu/ml | |
| 15 mins | 1.0 × 10⁵ ± 1.2 × | 1.0 × 10⁵ ± 4.3 × | 0 pfu/ml |
| | 10⁴ pfu/ml | 10⁴ pfu/ml | |
| 60 mins | 1.05 × 10⁵ ± 7.0 × | 1.0 × 10⁵ ± 4.9 × | 0 pfu/ml |
| | 10⁴ pfu/ml | 10⁴ pfu/ml | |
| 120 mins | 1.05 × 10⁵ ± 6.0 × | 1.0 × 10⁵ ± 5.3 × | 0 pfu/ml |
| | 10⁴ pfu/ml | 10⁴ pfu/ml | |
| 180 mins | 1.05 × 10⁵ ± 8.0 × | 9.0 × 10⁴ ± 9.1 × | 0 pfu/ml |
| | 10⁴ pfu/ml | 10³ pfu/ml | |

### Example 3

50 ml of 10,000 ppm NO was injected into a sterile IV bag containing 50 ml of 0.9% saline solution. From stock of influenza A%Victoria/H3N2 an inoculum of 10⁷ virions was prepared in phosphate buffered saline (PBS).

100 μl of the inoculum was dried on a glass microscope slide. The virus was the reconstituted using 900 μl of NO-saline (nitrisol) or 900 μl of PBS. The reconstituted virus was then inoculated onto 6-well trays of confluent MDCK cells. A standard plaque assay was performed. After 2 days the plates were fixed and stained. The plaques were counted and the results are shown in Table 3. As can be seen the NO-treated saline reduced the number of infectious virions by 2-3 logs compared to the samples reconstituted in PBS.

TABLE 3
Effect of NO-treated saline on reconstituted Influenza A/Victoria/H3N2

| | PBS | NO-treated saline | |
|---|---|---|---|
| 0 mins | 1 × 10⁷ pfu/ml | 1 × 10⁷ | pfu/ml |
| 5 mins | 1 × 10⁵ pfu/ml | 1 × 10³ | pfu/ml |
| 10 mins | 1 × 10⁵ pfu/ml | 100 | pfu/ml |
| 15 mins | 1 × 10⁵ pfu/ml | 75 | pfu/ml |

1. Use of nitric oxide for inhibiting the transmissibility of a virus.

2. Use according to claim 1 wherein the nitric oxide is in gaseous form.

3. Use according to claim 1 wherein the nitric oxide is in solution.

4. Use according to according to claim 1 wherein the nitric oxide is dissolved in water.

5. An anti-viral composition comprising a nitric oxide solution.

6. A composition according to claim 5 wherein the solvent is selected from water, alcohol, and combinations thereof.

7. A composition according to claim 5 wherein the solvent is saline.

8. A composition according to claim 5 wherein the pH of the solution is from about 3 to about 6.

9. A composition according to claim 5 wherein the concentration of nitric oxide is from about 10 μM to about 200 μM.

10. An anti-viral spray or aerolizing device comprising an orifice and a reservoir wherein the reservoir communicates with the orifice and comprises a supply of nitric oxide.

11. A device according to claim 10 wherein the nitric oxide is in the form of a compressed gas.

12. A device according to claim 10 wherein the reservoir comprises a solution of nitric oxide.

13. A device according to claim 10 wherein the reservoir comprises a solution of nitric oxide, the solution having pH of from about 3 to about 6.

14. A device according to claim 10 wherein the reservoir comprises a solution of nitric oxide, the concentration of nitric oxide being from about 10 μM to about 200 μM.

15. An anti-viral air-filter comprising a filter element and nitric oxide.

16. An air-filter according to claim 15 comprising a dischargeable supply of gaseous nitric oxide.

17. An air-filter according to claim 15 comprising a dischargeable supply of gaseous nitric oxide which periodically delivers nitric oxide in a concentration of at least about 25 parts per million.

18. An air-filter according to claim 15 wherein the nitric oxide is in the form of a solution.

19. An air-filter according to claim 15 wherein the nitric oxide is applied to the filter element in the form of a solution, the solution having a pH of from about 3 to about 6 and a nitric oxide concentration of from about 10 μM to about 200 μM.

20. An air-filter according to claim 15 wherein the filter element is selected from fibrous woven and non-woven fabrics.

21. An air-filter according to claim 15 wherein the filter is a facemask configured and sized to substantially cover the nose and mouth of the user and to be secured thereto.

22. An air-filter according to claim 15 wherein the filter is an air-duct filter configured and sized to fit in an air duct system.

23. An apparatus comprising:
a facemask configured to communicably cooperate with a supply of nitric oxide;
a dischargeable supply of nitric oxide; and
a device configured to controllably provide from the dischargeable supply from about 100 ppm to about 200 ppm of nitric oxide.

24. An apparatus according to claim 23, wherein the supply of nitric oxide is demountably engagable with the facemask.

25. A kit comprising:
a facemask configured and sized to substantially cover the nose and mouth of the user and to be secured thereto;
a dischargeable supply of nitric oxide;
the facemask and nitric oxide being sealably contained within a gas-impermeable container.

26. A method of inhibiting the transmission of viral microorganisms, the method comprising the steps of:
providing a supply of nitric oxide;
contacting the viral microorganisms with the nitric oxide.

27. The method according to claim 26 wherein the nitric oxide is in gaseous form in concentration of from about 25 parts per million to about 100 parts per million.

28. The method according to claim 26 wherein the nitric oxide is in solution in a concentration of from about 10 μM to about 200 μM.

29. The method of claim 26 wherein the viral microorganisms are selected from the group consisting of pathogenic influenza viruses.

30. A method for treating an animal having pathogenic viral microorganisms in the respiratory tract of the animal comprising the step of delivering by the inhalation route to the respiratory tract of the animal an amount of nitric oxide effective to inhibit the transmissibility of said pathogenic microorganisms.
